(19) United States
(12) Patent Application Publication
(10) Pub. No.: US 2007/0073414 A1
(43) Pub. Date: Mar. 29, 2007

(54) Assembly for controlling emptying of a female urine bladder, incontinence valve, and urine drainage assembly comprising the assembly

(76) Inventor: Patrik Westerkull, Hovas (SE)

Correspondence Address: Gauthier & Connors LLP;Suite 2300, 225 Franklin Street, Boston, MA, 02110, US

(21) Appl. No.: 11/525,596

(22) Filed: Sep. 22, 2006

Related U.S. Application Data
(63) Continuation of application No. PCT/SE05/00444, filed on Mar. 24, 2005.

(30) Foreign Application Priority Data
Mar. 24, 2004 (SE) 0400757-1

(51) Int. Cl. A61F 2/04

(52) U.S. Cl. 623/23.66; 600/30; 623/23.68

(57) ABSTRACT

An assembly for controlling emptying of a urine bladder, adapted to be positioned into a urethra of a female is disclosed. It comprises an oblong and substantially cylindrically shaped body to be inserted into the urethra, the body presenting a urine inlet and a urine outlet, at the ends of the body, connected by a through hole extending through the body substantially in the longitudinal direction of the body, the body presenting a length being shorter than the length of the urethra, causing the bladder neck to block the body from being in contact with the urine in the bladder when positioned in the urethra, the body presenting a length being longer than a cross section of the body, the through hole being able to receive a urine conducting member, which is projectable through the urine inlet to enter the bladder, a holder mounted to an outer surface of the body, and a plate limiting the movement in the longitudinal direction towards the urine inlet and fixed at the urine outlet, the plate presenting a larger cross section than the body. An incontinence valve, and a urine drainage assembly comprising the assembly are also disclosed.

### TECHNICAL FIELD

A first aspect of the present invention relates to an assembly for controlling emptying of a urine bladder.

A second aspect of the present invention relates to an incontinence valve comprising the assembly of the first aspect.

A third aspect of the present invention relates to a urine drainage assembly comprising the assembly of the first aspect.

### BACKGROUND OF INVENTION

The use of intra urethral incontinence valves represents an important treatment for female patients having incontinence problems. For severe incontinence the alternative is mayor surgery with significant risks and uncertain outcome. An intra urethral incontinence valve is therefore a treatment option where the patient can become independent of large size pads. Intra urethral incontinence valves are described in U.S. Pat. No. 3,812,841, WO 99/08629, U.S. Pat. No. 5,030,199 and U.S. Pat. No. 6,105,580.

A mayor challenge for intra urethral incontinence valves is the problem with blockage due to encrustations and formation of bladder stones on the surface of the portion of the device that is in the bladder and in direct contact with the urine most of the time. It is important that these devices offer a complete emptying of the bladder since residual urine In the bladder is a well-known cause for urinary tract infections. Therefore the design in both WO 99/08629 and U.S. Pat. No. 5,030,199 includes a valve member reaching all the way from the bladder to the opening of the urethra. These designs offer complete emptying of the bladder but on the other hand they involve a significant risk of encrustations on the foreign body constantly seated in the bladder. In both U.S. Pat. No. 3,812,841 and U.S. Pat. No. 6,105,580 a design for a male urethra is described. These designs however do present limitations since there is a significant risk of incomplete emptying of the bladder and urinary tract infections, especially, this would be the case if such a design would be used for the female urethra being much shorter than the male urethra. In spite of several efforts since U.S. Pat. No. 3,812,841 was filed in 1972, incomplete emptying of the bladder, encrustations and urinary tract infections are still unsolved challenges with these devices.

The use of indwelling catheters also called Foley catheters used for urine drainage is very common for some patient groups. The device consists of an indwelling balloon and a catheter tube that reaches from the bladder to a urine-collecting bag mounted on the bedside or on the patient's leg. The function of the balloon is to keep the catheter in place. A significant problem with these catheters is the encrustations and bladder stones that are formed on the balloon in the bladder. Irritation on the bladder and urinary tract infections are common side effects of these devices, still they are frequently used since alternatives are lacking. The cost of indwelling catheters is quite significant due to the integrated design with balloon and catheter in one unit.

### SUMMARY OF INVENTION

The present invention offers an efficient solution to the problems with the current designs of intra urethral incontinence valves and indwelling catheters.

According to the first aspect of the present invention, it deals with an assembly for controlling emptying of a urine bladder is disclosed. The assembly is adapted to be positioned into a urethra of a female. The assembly comprises an oblong and substantially cylindrically shaped body to be inserted into the urethra. The body presents a urine inlet and a urine outlet, at the ends of the body. The outlets are connected by a through hole extending through the body substantially in the longitudinal direction of the body. The body presents a length being shorter than the length of the urethra. Further, the body presents a length being longer than a cross section of the body. The through hole is able to receive a urine-conducting member, which is projectable through the urine inlet to enter the bladder and may be connected to a urine collecting bag. The assembly further comprises a holder mounted to the outer surface of the body and a plate limiting the movement in the longitudinal direction towards the urine inlet and fixed at the urine outlet, the plate presenting a larger cross section than the body.

The holder is responsive for keeping the assembly in place in the urethra.

The plate that limits the movement in the longitudinal direction towards the urine inlet leads to the advantage of a hindering the assembly from moving too far into the urethra, resulting in that the urine inlet, or even the whole assembly, ends up in the bladder.

That the body presents a length being shorter than the length of the urethra combined with the plate limiting the movement leads to the advantage that the body will not be in contact with the urine in the bladder since it is only the urine-conducting member that will be entered into the bladder. Thus, no part of the assembly is in the bladder.

The length of the female urethra often lies in the interval 35-70 mm, resulting in a length of the body lying in the interval approx. 20-60 mm will open up for a possibility of offering a body that presents a length that will lead to the advantage that the body will not be in the bladder. This decreases the risk of formation of encrustations and stones, etc. on the body, or the assembly, since the contact with urine is limited. The closing function of the bladder neck assists in limiting the contact between urine and the body, or the assembly. In case the length of the urethra is known then an assembly presenting a body having a suitable length may be used. In case the length of the urethra is unknown then an assembly presenting a length that lies in the lower end of the interval 20-60 mm may be used.

That the body presents a length being longer than a cross section of the body results in the advantage that it is easier to insert the assembly into to the female urethra since the there is a decreased risk of tilting the assembly in the urethra when inserting it. There is also a decreased risk of penetrating the urethra wall with the urine-conducting member.

Thus, the present invention includes a body with a holder in the urethra, the body being shorter than the urethra, which minimizes the risk of encrustations on this portion of the device since it is designed in a way that it does not reach into the bladder.

Significant benefits compared with hitherto known designs can be reached since a complete emptying of the bladder can be done but still the risks of encrustations and stone formation is minimal.

In a preferred embodiment, the length of the body is longer than 1.5 times the cross section of the body. This results in the advantage that it is even easier to insert the assembly into to the female urethra since the there is an even more decreased risk of tilting the assembly in the urethra when inserting it. There is also a further decreased risk of penetrating the urethra wall.

In a preferred embodiment, the length of the body is longer than 2 times the cross section of the body. This results in the advantage of an even further ease when inserting the assembly into to the female urethra since the risk of tilting the assembly in the urethra is even more decreased when inserting it. There is also an even further decreased risk of penetrating the urethra wall.

In a preferred embodiment, the outer surface of the body presents an R_{A} lower than 10. A smooth surface offers the advantage of decreasing the risk for encrustations and bacterial collection on the surface of the body.

In a preferred embodiment, the holder comprises a shape memory compound, or alloy, such as a bi metal or a stent.

Thus an advantage of the assembly is that the body is less likely to accidentally come out the urethra and the plate blocks the body from reaching the bladder.

In a preferred embodiment, the assembly further comprises a removable closing means fixable at the urine outlet. This offers the advantage of being able to close the outlet resulting in decreased risk of leakage.

In a preferred embodiment, the assembly further comprises a manually operated valve, such as a non-return valve or a duck valve.

In a preferred embodiment, the manually operated valve is at least one of in the through hole, at the urine inlet, and at the urine outlet. An advantage of the valve being in the through hole is that the risk of the valve coming loose from the assembly is lower since the design will keep the valve in place. An advantage of the valve being at the urine inlet, or at the urine outlet, is that such an assembly is easier and cheaper to manufacture.

In a preferred embodiment of the assembly, it further comprises a cover sleeve mounted in the body and covering the flushing holes. The purpose of the flushing holes in the body is to facilitate flushing of shape memory compounds, such as stents. The cover sleeve offers the advantage of preventing tissue from growing in through the flushing holes of the body.

In a preferred embodiment, the assembly further comprises a removable closing means fixable at the urine outlet. This offers the advantage of providing an extra closing capability.

According to the second aspect, an incontinence valve comprising the assembly is disclosed. The incontinence valve further comprises the urine-conducting member, which is projectable via the through hole at the urine inlet. The urine-conducting member presents a length sufficient to enter the bladder, when projected. This offers the advantage of an incontinence valve that has no part constantly in the bladder but only during emptying of the bladder. The incontinence valve comprises a urine-conducting member that can pass through the above-mentioned body and reaching all the way into the bladder during emptying of the bladder. When not emptying the bladder this urine-conducting member is however not reaching into the bladder and the above mentioned body is closed for urine flow.

In a preferred embodiment, the manually operated valve is constituted by the telescopic urine conducting member, which, when projecting through the urine inlet, allows emptying the bladder, and otherwise closes off the through hole for urine flow.

This offers a simple and safe design for controlling the flow of urine through the incontinence valve.

In a preferred embodiment, the urine-conducting member further comprises releasable fastening means for withdrawing the urine-conducting member. This offers the advantage that after having emptied the bladder, the releasable fastening means offers an opportunity of releasable fastening, e.g. a tube to the inner side of the urine conducting member, and thereafter pulling the urine conducting member back into the through hole and thereafter loosening the tube by a slight pull. The tube may be connected to a urine collecting bag.

In a preferred embodiment, the incontinence valve comprises a removable closing means fixable at the urine outlet. This offers the advantage of providing an extra closing capability.

In a preferred embodiment, the incontinence valve comprises a flexible female connector on the closing means and a rigid male connector portion at the urine outlet.

In a preferred embodiment, the incontinence valve comprises the assembly of the first aspect and further comprises a flexible male connector on the closing means and a rigid female connector portion at the urine outlet.

The presence of the flexible female/male connector on the closing means and a rigid male/female connector portion at the urine outlet in case of an incontinence valve, leads to the advantage of securing the fixation of the removable closing means. In a preferred embodiment, the interconnection is designed as a press fit. This press fit may then also work as a seal for fluid leakage at the interconnection. This design is a cost efficient version of a fixation. It is important that the fixation is sufficiently stable so that the removable closing means does not fall off from the assembly. It is however also important that the fixation force is lower than the force needed to pull the assembly out of the urethra. It is preferred that this fixation is not mainly depending on the surface friction at the interconnection since friction may vary dependent on if the surface is wet or dry. A flexible female connection where the fixation force is dependent on the spring force of the flexible female connection is less dependent on the surface friction. An alternative solution is a flexible male connection that connects to a rigid female connection portion on the assembly. An alternative solution is a flexible male connection that connects to a rigid female connection portion on the assembly. A flexible male or female connection can be combined with a separate leakage sealing.

According to the third aspect, a urine drainage assembly comprising the assembly is disclosed. The urine drainage assembly offers the advantage of a lower risk of urinary tract infection. As an alternative to an indwelling catheter and in a preferred embodiment, it comprises a urine-conducting member that can pass through the above-mentioned body and reach all the way into the bladder to drain the bladder. Since this urine-conducting member is kept in place with the holder of the above-mentioned body, no balloon is needed to keep this urine-conducting member in place. The urine-conducting member of the present invention can be manufactured to a significantly lower cost than the cost of a traditional indwelling catheter. A significant advantage with the present invention is that there is no balloon in the bladder causing irritation of the bladder.

In a preferred embodiment, the urine drainage assembly comprises the assembly of the first aspect.

In a preferred embodiment, the urine drainage assembly comprises a removable closing means fixable at the urine outlet. This offers the advantage of providing an extra closing capability.

In a preferred embodiment the urine drainage assembly comprises a flexible female connector on the urine conducting member and a rigid male connector portion at the urine outlet.

In a preferred embodiment, the urine drainage assembly comprises a flexible male connector on the urine conducting member and a rigid female connector portion at the urine outlet.

The presence of the flexible female/male connector on the urine conducting member and a rigid male/female connector portion at the urine outlet in case of a urine drainage assembly, leads to the advantage of securing the fixation of the urine conducting member. In a preferred embodiment, the interconnection is designed as a press fit. This press fit may then also work as a seal for fluid leakage at the interconnection. This design is a cost efficient version of a fixation. It is important that the fixation is sufficiently stable so that the end of the urine conducting member placed in the bladder does not slip out of the bladder since this would cause the draining of the bladder to stop. It is however also important that the fixation force is lower than the force needed to pull the assembly out of the urethra. It is an advantage if this fixation is not mainly depending on the surface friction at the interconnection since friction may vary dependent on if the surface is wet or dry. A flexible female connection where the fixation force is dependent on the spring force of the flexible female connection is less dependent on the surface friction. An alternative solution is a flexible male connection that connects to a rigid female connection portion on the assembly. A flexible male or female connection can be combined with a separate leakage sealing.

A key advantage of the three aspects is that the emptying may be effectuated using a tube to be inserted through the through hole and further up the urethra, passing the bladder neck and opening the bladder causing the bladder to be emptied. The tube may be connected to a urine-collecting bag. In the case of the incontinence valve having a telescopic urine conducting member, a stick may be sufficient to push the urine conducting member to its projected position at which emptying of the bladder is achieved. This also allows a more efficient emptying of the bladder since the urine-conducting member may be entered into the bladder to allow residual urine to be emptied.

### DESCRIPTION OF DRAWINGS

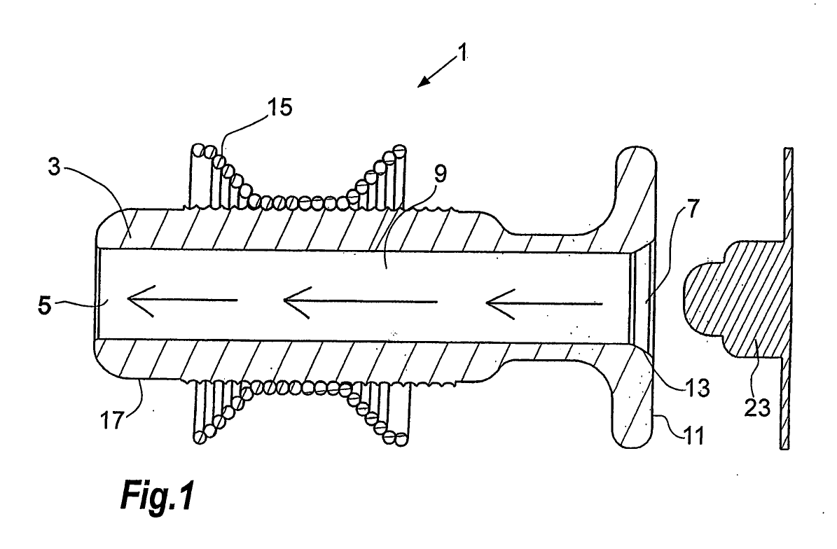

In FIG. 1, an embodiment of an assembly for controlling emptying of a urine bladder, adapted to be positioned into a urethra of a female is disclosed. In FIG. 1, the arrows indicate the direction of insertion of a urine-conducting member. The direction is opposite to the direction of the urine flow during emptying.

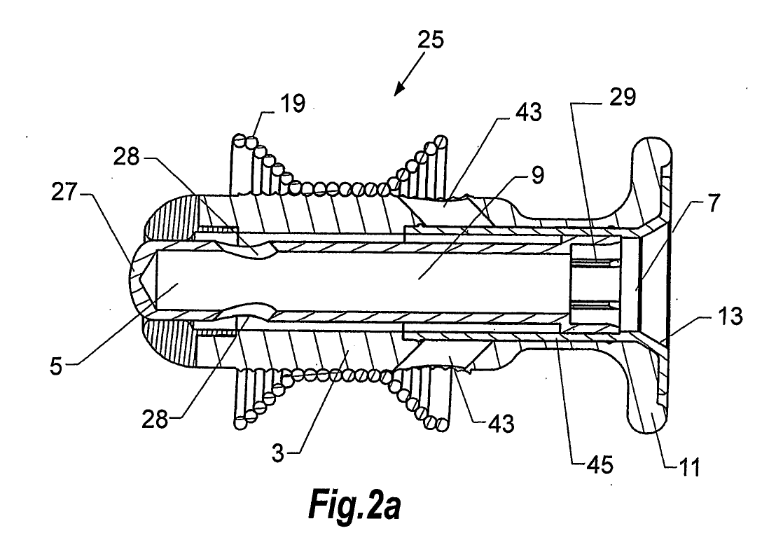

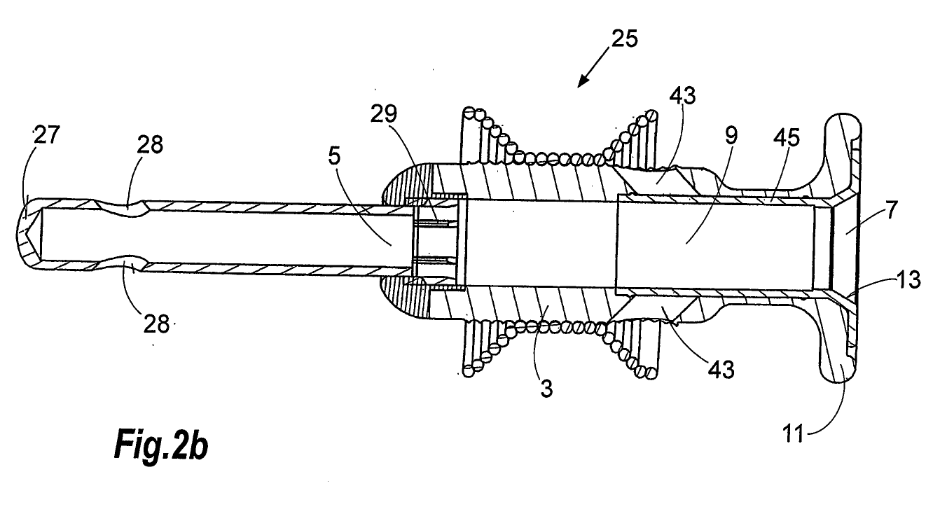

In FIGS. 2A and 2B, an embodiment of an incontinence valve is disclosed.

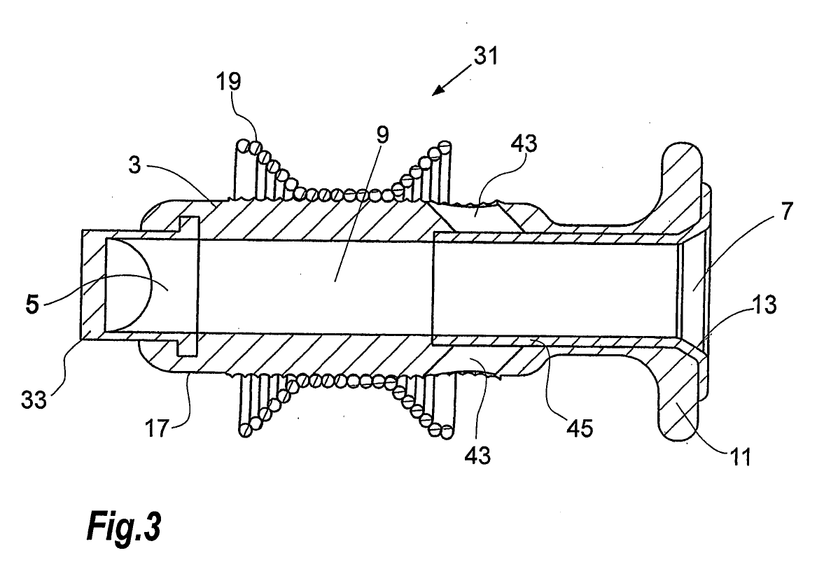

In FIG. 3, an embodiment of a urine drainage assembly is disclosed.

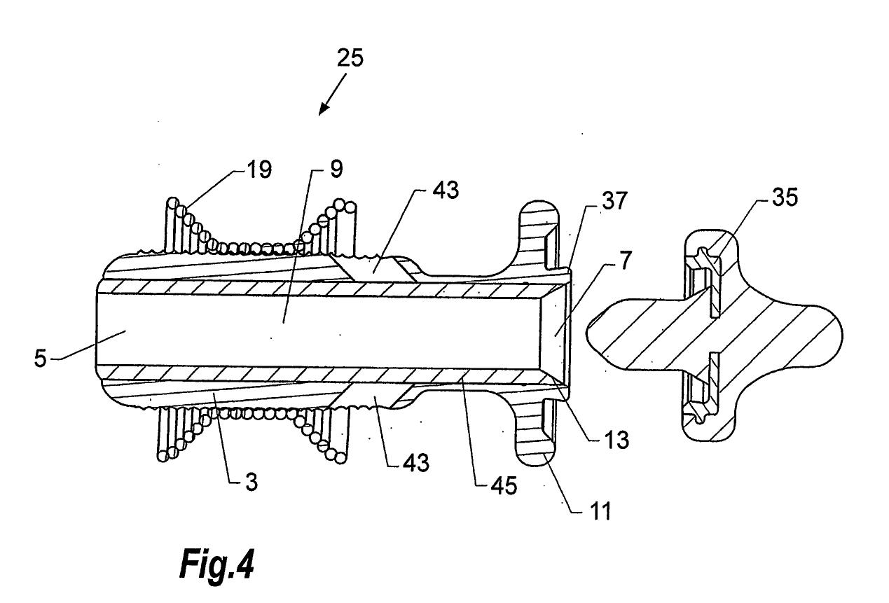

In FIG. 4 an embodiment of an incontinence valve is disclosed.

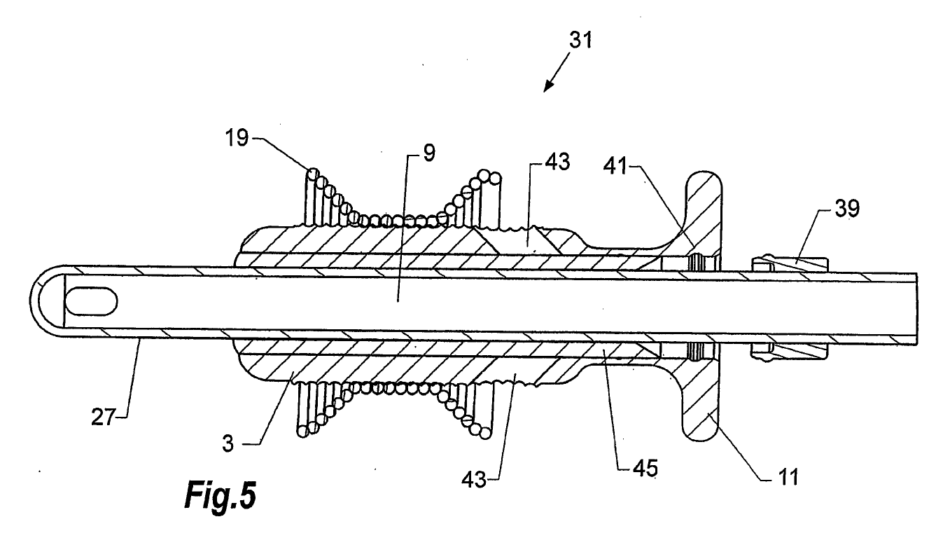

In FIG. 5 an embodiment of a urine drainage assembly is disclosed.

### DESCRIPTION OF PREFERRED EMBODIMENTS

In FIG. 1, an assembly 1 for controlling emptying of a urine bladder, adapted to be positioned into a urethra of a female is disclosed. It comprises an oblong and substantially cylindrically shaped body 3 to be inserted into the urethra. The body 3 presents a urine inlet 5 and a urine outlet 7, which are located at the ends of the body 3. The inlet 5 and the outlet 7 are connected to each other by a through hole 9 extending through the body 3 substantially in the longitudinal direction of the body 3. Thus, the through hole 9 provides a passage for urine.

The body 3 presents a length that is shorter than the length of the urethra. This causes the bladder neck to block the body from being in contact with the urine in the bladder when positioned in the urethra, since a plate 11, which will be elaborated further below, blocks insertion of the assembly too long into the urethra. The body 3 presents a length that is longer than a cross section of the body 3.

The through hole 9 is able to receive a urine-conducting member, which is projectable through the through hole 9 and out through the urine inlet 5 to enter the bladder. The purpose of the urine-conducting member is to open the bladder allowing emptying of the bladder. This is indicated in FIG. 1 by the arrows in the through hole 9. Thus, the arrows indicate the direction of insertion of the urine-conducting member. The direction is opposite to the direction of the urine flow during emptying. In a preferred embodiment, the urine conducting member is a tube, which is inserted, when the bladder is to be emptied, into the urethra, through the urine outlet 7, through the through hole 9, and out through the urine inlet 5 and up into the bladder and urine will flow through the tube.

In a preferred embodiment, the urine outlet 7 is provided with a conical arrangement 13 facilitating the insertion of the urine-conducting member, providing a secure insertion thereof since the risk of it pushing the assembly 1 up the urethra decreases.

The assembly 1 further comprises a holder 15 mounted to the outer surface 17 of the body 3, which in a preferred embodiment comprises a shape memory compound, such as a stent 19.

The assembly 1 further comprises a plate 11, as indicated above, limiting the movement in the longitudinal direction towards the urine inlet 5 and fixed at the urine outlet 7. The plate 11 presents a larger cross section than the body 3. In a preferred embodiment, the plate 11 presents a hole that is connected with the through hole 9.

In a preferred embodiment, the assembly 1 wherein the outer surface 17 of the body 3 presents an R_{A} lower than 10.

In a preferred embodiment, the assembly 1 further comprises a removable closing means 23, such as a plug or a stopper, which is removably fixable at the urine outlet 7.

In FIG. 2A, an incontinence valve 25 is disclosed that comprises the assembly 1 and a manually operated valve located in the through hole 9, wherein the manually operated valve is constituted by the operation of a urine-conducting member 27 in relation to the through hole 9.

In a preferred embodiment, the urine-conducting member 27 is projectable via the through hole 9 at the urine inlet 5. It should be pointed out that the urine-conducting member 27 is not necessarily fully housed in the through hole 9 when the valve is closed but a portion thereof may project out through the urine inlet 5. The manually operated valve may be constituted by the telescopic urine conducting member 27, which, when projecting through the urine inlet 5, allows emptying the bladder, and otherwise closes off the through hole 9 for urine flow. In FIG. 2B, an incontinence valve 25 and a urine-conducting member 27 in its projected state is disclosed. The urine conducting member 27 may be activated by one of a tube or a stick, which is inserted into the urethra when to empty the bladder. The tube or the stick contacts the urine-conducting member 27 and is responsive for projecting it through the urine inlet 5 into the bladder. The tube presents an advantage over the stick since the tube offers the advantage of a more user-friendly solution since the tube will lead the urine to a place suited for receiving urine, such as a toilet. A stick may activate the incontinence valve 25, however there may be a risk of the urine spilling.

In a preferred embodiment, the top of the urine-conducting member presents a number of holes 28 through which the urine will flow. There may be a number of holes 28 or just a single one. The locations of the holes are in a preferred embodiment on the side near the top of the urine-conducting member 27. In another embodiment, a single hole is located on the top of the urine-conducting member 27.

In a preferred embodiment, the urine-conducting member 27 further comprises releasable fastening means 29 for withdrawing the urine-conducting member 27. The term releasable fastening means 29 includes e.g. releasable hooking means. Withdrawing the urine-conducting member 27 out from the bladder, at least partially, into the urine inlet 5 may be accomplished by the same means that was used to projected it. Preferred embodiments of releasable fastening means 29 include an inside of the urine conducting member 27 that presents a narrowing cross section which makes it possible for a tube or a stick to become releasably fixed thereto. After having moved the urine-conducting member 27 from its projecting position, the tube, or the stick, is loosened by pulling it lightly.

In a preferred embodiment, the assembly further comprises a removable closing means fixable at the urine outlet.

In a preferred embodiment, a urine drainage assembly 31 is disclosed in FIG. 3. In a preferred embodiment, the urine drainage assembly 31 comprises a duck valve 33 arranged at the urine inlet 5.

In a preferred embodiment where the urine conducting member 27 and the removable closing means 23 include a flexible female connection 35 that can be connected to a rigid male connection portion 37 at the urine outlet 7 of the body 3. In FIG. 4 an embodiment of an incontinence valve 25 having a rigid male connection portion 37 on which a flexible female connection 35 on the removable closing means 23 can be attached.

In a preferred embodiment where the urine conducting member 27 and the removable closing means 23 include a flexible male connection 39 that can be connected to a rigid female connection portion 41 at the urine outlet 7 of the body 3. In FIG. 5 an embodiment of a urine drainage assembly 31 having a rigid female connection portion 41 on which a flexible male connection 39 on the urine conducting member 27 can be attached.

In a preferred embodiment flushing holes 43 through the body 3 facilitates the flushing of the stents 19. A cover sleeve 45 is mounted in the body 3 to block the flushing holes 43.

1. An assembly for controlling emptying of a urine bladder, adapted to be positioned into a urethra of a female, comprising:
and oblong and substantially cylindrically shaped body to be inserted into the urethrea,
the body presenting a urine inlet and a urine outlet, at the ends of the body, connected by a through hole extending through the body substantially in the longitudinal direction of the body,
the body presenting a length being shorter than the length of the urethra, causing the bladder neck to block the body from being in contact with the urine in the bladder when positioned in the urethra,
the body presenting a length being longer than a cross section of the body,
the through hole being able to receive a urine conducting member, which is projectable through the urine inlet to enter the bladder,
a holder mounted to an outer surface of the body, and
a plate limiting the movement in the longitudinal direction towards the urine inlet and fixed at the urine outlet, the plate presenting a larger cross section than the body.

2. An assembly according to claim 1, wherein the length of the body is longer than 1.5 times the cross section of the body.

3. An assembly according to claim 1, wherein the length of the body is longer than 2 times the cross section of the body.

4. An assembly according to claim 1, wherein the outer surface of the body presents and R_{A} lower than 10.

5. An assembly according to claim 1, wherein the holder comprises a shape memory compound.

6. An assembly according to claim 1, further comprising a manually operated valve.

7. An assembly according to claim 6, wherein the manually operated valve is constituted by a non-return valve.

8. An assembly according to claim 6, wherein the manually operated valve is constituted by a duck valve.

9. An assembly according to claim 6, wherein the manually operated valve is at least one of:
in the through hole,
at the urine inlet, and
at the urine outlet.

10. An assembly according to claim 1, further comprising a cover sleeve mounted in the body and covering flushing holes.

11. An incontinence valve comprising the assembly according to claim 1, further comprising the urine conducting member being projectable via the through hole at the urine inlet.

12. An incontinence valve according to claim 11, wherein the manually operated valve is constituted by a telescopic urine conducting member, which, when projecting through the urine inlet, allows emptying the bladder, and otherwise closes off the through hole for urine flow.

13. An incontinence valve according to claim 12, wherein the urine conducting member further comprises releasable fastening means for withdrawing the urine conducting member.

14. An assembly according to claim 12, further comprising a removable closing means fixable at the urine outlet.

15. An incontinence valve according to claim 14, further comprising a flexible female connector on the closing means and a rigid male connector portion at the urine outlet.

16. An incontinence valve according to claim 14, further comprising a flexible male connector on the closing means and a rigid female connector portion at the urine outlet.

17. A urine drainage assembly comprising the assembly according to claim 1.

18. A urine drainage assembly according to claim 17, further comprising a urine conducting member.

19. A urine drainage assembly according to claim 18, further comprising a flexible female connector on the urine conducting member and a rigid male connector portion at the urine outlet.

20. A urine drainage assembly according to claim 18, further comprising a flexible male connector on the urine conducting member and a rigid female connector portion at the urine outlet.
